(19) United States
(12) Patent Application Publication
(10) Pub. No.: US 2021/0096934 A1
(43) Pub. Date: Apr. 1, 2021

(54) SYSTEMS AND METHODS FOR ENHANCING A PATIENT POSITIONING SYSTEM

(71) Applicant: SHANGHAI UNITED IMAGING INTELLIGENCE CO., LTD., Shanghai (CN)

(72) Inventors: ABHISHEK SHARMA, Boston, MA (US); ARUN INNANJE, Lexington, MA (US); ZIYAN WU, Lexington, MA (US)

(21) Appl. No.: 16/589,656

(22) Filed: Oct. 1, 2019

(51) Int. Cl. G06F 9/54; G06N 3/04; G06N 3/08; G06N 20/20; G06T 7/00; G06T 1/00

(52) U.S. Cl. CPC G06F 9/542; G06N 3/0454; G06N 3/08; G06T 2207/20081; G06T 7/0012; G06T 1/0007; G06T 2207/20084; G06N 20/20

(57) ABSTRACT

Methods and systems for using a medical imaging apparatus for acquiring a medical image. For example, a computer-implemented method for using a medical imaging apparatus for acquiring a medical image of a patient includes: determining a first positioning instruction by a first neural network, acquiring a first image based on the first positioning instruction; receiving the first image; identifying one or more first features associated with the acquired first image; determining a first quality assessment based on the identified one or more first features; generating a first feedback based on the first quality assessment; receiving the first feedback by the first neural network; and changing one or more first parameters of the first neural network based on the first feedback.

### 1. BACKGROUND OF THE INVENTION

Certain embodiments of the present invention are directed to positioning an object. More particularly, some embodiments of the invention provide methods and systems for positioning a patient. Merely by way of example, some embodiments of the invention have been applied to enhancing a patient positioning system. But it would be recognized that the invention has a much broader range of applicability.

Conventional patient positioning systems, such as ones for medical CT, MR, X-ray, or ultrasound scanners, are prone to errors in estimating the degree of deviation of a patient pose from a reference pose. Owing to such errors, multiple scans are often needed to produce a satisfactory scan. Once deployed in a hospital, conventional patient positioning systems rely on manual review and annotation to identify errors in the medical scans produced by the scanners. Some patient positioning systems may receive infrequent updates, such as monthly or quarterly updates, to incorporate manual feedbacks to help reduce estimating errors. Such procedure is inefficient and therefore it is desirable to have a method and a system for enhancing a patient positioning system with greater efficiency to improve patient positioning.

### 2. BRIEF SUMMARY OF THE INVENTION

Certain embodiments of the present invention are directed to positioning an object. More particularly, some embodiments of the invention provide methods and systems for positioning a patient. Merely by way of example, some embodiments of the invention have been applied to enhancing a patient positioning system. But it would be recognized that the invention has a much broader range of applicability.

In various embodiments, a computer-implemented method for using a medical imaging apparatus for acquiring a medical image of a patient includes: receiving a scanning protocol; determining a first positioning instruction based at least in part on the scanning protocol by a first neural network, the first neural network being previously-trained for positioning; acquiring a first image based at least in part on the first positioning instruction and the scanning protocol; receiving the first image; identifying one or more first features associated with the acquired first image; determining a first quality assessment based at least in part on the identified one or more first features, the first quality assessment being associated with the first image; generating a first feedback based at least in part on the first quality assessment; receiving the first feedback by the first neural network; and changing one or more first parameters of the previously-trained first neural network based at least in part on the first feedback. In certain examples, the computer-implemented method is performed by one or more processors.

In various embodiments, a system for computer-implemented method for using a medical imaging apparatus for acquiring a medical image of a patient includes: a protocol receiving module configured to receive a scanning protocol; an instruction determining module configured to determine a first positioning instruction based at least in part on the scanning protocol by a first neural network, the first neural network being previously-trained for positioning; an image acquiring module configured to acquire a first image based at least in part on the first positioning instruction and the scanning protocol; an image receiving module configured to receive the first image; a feature identifying module configured to identify one or more first features associated with the acquired first image; a quality assessment module configured to determine a first quality assessment based at least in part on the identified one or more first features, the first quality assessment being associated with the first image; a feedback generating module configured to generate a first feedback based at least in part on the first quality assessment; a feedback receiving module configured to receive the first feedback by the first neural network; and a parameter changing module configured to change one or more first parameters of the previously-trained first neural network based at least in part on the first feedback.

In various embodiments, a non-transitory computer-readable medium with instructions stored thereon, that when executed by a processor, perform the processes including: receiving a scanning protocol; determining a first positioning instruction based at least in part on the scanning protocol by a first neural network, the first neural network being previously-trained for positioning; acquiring a first image based at least in part on the first positioning instruction and the scanning protocol; receiving the first image; identifying one or more first features associated with the acquired first image; determining a first quality assessment based at least in part on the identified one or more first features, the first quality assessment being associated with the first image; generating a first feedback based at least in part on the first quality assessment; receiving the first feedback from the second neural network by the first neural network; and changing one or more first parameters of the previously-trained first neural network based at least in part on the first feedback.

In various embodiments, a method for using a medical imaging apparatus for acquiring a medical image of a patient includes: receiving a scanning protocol; determining a first positioning instruction based at least in part on the scanning protocol by a first neural network, the first neural network being previously-trained for positioning; acquiring a first image based at least in part on the first positioning instruction and the scanning protocol; receiving the first image; identifying one or more first features associated with the acquired first image; determining a first quality assessment based at least in part on the identified one or more first features, the first quality assessment being associated with the first image; generating a first feedback based at least in part on the first quality assessment; receiving the first feedback by the first neural network; and changing, by the first neural network, one or more first parameters of the previously-trained first neural network based at least in part on the first feedback.

Depending upon embodiment, one or more benefits may be achieved. These benefits and various additional objects, features and advantages of the present invention can be fully appreciated with reference to the detailed description and accompanying drawings that follow.

### 3. BRIEF DESCRIPTION OF THE DRAWINGS

FIG. 1 is a simplified diagram showing a system for using a medical imaging apparatus for acquiring a medical image of a patient, according to some embodiments.

FIG. 2 is a simplified diagram showing a method for using a medical imaging apparatus for acquiring a medical image of a patient, according to some embodiments.

FIG. 3 is a simplified diagram showing a method for using a medical imaging apparatus for acquiring a medical image of a patient, according to some embodiments.

FIG. 4 is a simplified diagram showing a computing system, according to some embodiments.

FIG. 5 is a simplified diagram showing a neural network, according to some embodiments.

### 4. DETAILED DESCRIPTION OF THE INVENTION

Certain embodiments of the present invention are directed to positioning an object. More particularly, some embodiments of the invention provide methods and systems for positioning a patient. Merely by way of example, some embodiments of the invention have been applied to enhancing a patient positioning system. But it would be recognized that the invention has a much broader range of applicability.

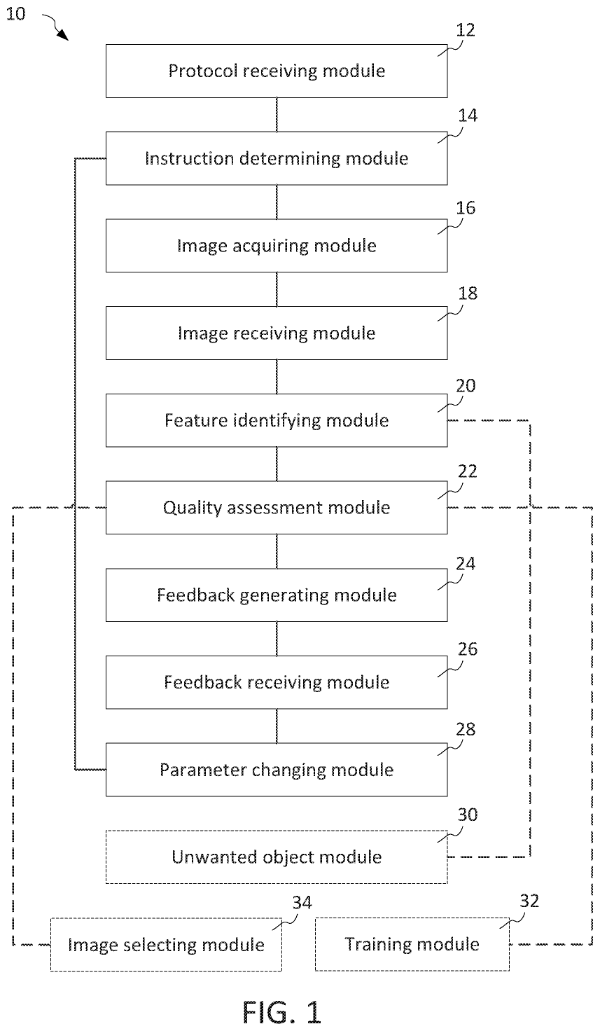

FIG. 1 is a simplified diagram showing a system for using a medical imaging apparatus for acquiring a medical image of a patient, according to some embodiments of the present invention. This diagram is merely an example, which should not unduly limit the scope of the claims. One of ordinary skill in the art would recognize many variations, alternatives, and modifications. In some examples, the system 10 includes a protocol receiving module 12, an instruction determining module 14, an image acquiring module 16, an image receiving module 18, a feature identifying module 20, a quality assessment module 22, a feedback generating module 24, a feedback receiving module 26, and a parameter changing module 28. In certain examples, the system 10 further includes an unwanted object module 30, a training module 32, and/or an image selecting module 34. In various examples, the system 10 is configured to enhance a patient positioning system and/or is a patient positioning system configured to enhance itself. Although the above has been shown using a selected group of components, there can be many alternatives, modifications, and variations. For example, some of the components may be expanded and/or combined. Other components may be inserted to those noted above. Depending upon the embodiment, the arrangement of components may be interchanged with others replaced.

In various embodiments, the protocol receiving module 12 is configured to receive a scanning protocol, such as a scanning protocol selected by a user. In some examples, the scanning protocol includes a scan type, a patient body type, a target body part, a sampling mask, a magnification, a working distance, a resolution, and/or a scanning rate. In various examples, the scanning protocol is selected from a menu, such as via a user interface.

In various embodiments, the instruction determining module 14 is configured to determine a positioning instruction based at least in part on the scanning protocol (e.g., one received by the protocol receiving module 12). In certain examples, the instruction determining module 14 includes a positioning neural network or is configured to use a positioning neural network. In some examples, the positioning neural network is a neural network trained, such as previously trained, for positioning an object. In certain examples, the object is a part of a patient. In certain examples, the instruction determining module 14 is configured to determine the positioning instruction based at least in part on a relative position between a patient position (e.g., a position of a target) and a reference position. For example, the positioning instruction includes guidance for adjusting the position of an imaging system (e.g., a scanning probe) and/or guidance for adjusting the position of the patient or a part of the patient. In some examples, the patient position is acquired based at least in part on a patient image acquired by the imaging system. In certain examples, the reference position is selected based at least in part on the scanning protocol and/or patient information. In some examples, the instruction determining module 14 is further configured to determine a target region based at least in part on the scanning protocol. For example, a target region includes a body part and/or a body organ. In certain examples, the instruction determining module 14 is further configured to determine a scanning technique (e.g., based at least in part on the scanning protocol) and to determine a scanning path based at least in part on the scanning technique.

In various embodiments, the image acquiring module 16 is configured to acquire an image based at least in part on the positioning instruction (e.g., determined by the instruction determining module 14) and/or the scanning protocol (e.g., received by the protocol receiving module 12). In certain examples, the image acquiring module 16 is configured to send the positioning instruction to a medical imaging apparatus (e.g., a scanning machine), such as to a positioning system (e.g., a robotic scanning platform and/or a robotic arm) of the medical imaging apparatus, for positioning a target (e.g., a patient). In some examples, the image acquiring module 16 is configured to send an imaging instruction to an imaging system (e.g., a scanning probe) to acquire an image according to the scanning protocol. In various examples, the image acquiring module 16 is configured to acquire an image by selecting the image from a pre-generated image database including one or more images previously acquired.

In various embodiments, the image receiving module 18 is configured to receive an image. For example, the image receiving module 18 is configured to receive an image by a quality assessment neural network, such as by a neural network trained (e.g., previously trained) for quality assessment. In some examples, the image receiving module 18 is configured to input an image into the quality assessment neural network for quality assessment. In certain examples, quality assessment is referred to as quality assurance.

In various embodiments, the feature identifying module 20 is configured to identify one or more features associated with an image, such as an image acquired by the image acquiring module 16. In some examples, the feature identifying module 20 is configured to identify one or more features associated with an image, such as using a neural network trained for identifying and/or extracting one or more features. In certain examples, the neural network trained for identifying one or more features is the same neural network trained for quality assessment. In various examples, the feature identifying module 20 is configured to identify, for example as a feature, a landmark, a visual feature, a geometric shape, and/or an unwanted object.

In various embodiments, the quality assessment module 22 is configured to determine a quality assessment based at least in part on one or more features, such as one or more features identified by the feature identifying module 20, by the neural network trained for identifying one or more features, and/or by the neural network trained for quality assessment. In some examples, a quality assessment is associated with an image, such as an image from which the one or more features are identified. In certain examples, a quality assessment is a quality score, such as a number from zero to one. In some examples, the quality assessment module 22 is configured to compare the identified one or more features (e.g., identified by the feature identifying module 20) against a target feature list and identify one or more missing features. In various examples, the target feature list includes one or more target features, which if identified to be in an image, help contributes to a satisfactory quality assessment. For example, the more target features identified from an image, the higher a quality score for the image.

In various embodiments, the feedback generating module 24 is configured to generate a feedback based at least in part on a quality assessment, such as a quality assessment determined by a quality assessment neural network and/or by a neural network trained (e.g., previously trained) for quality assessment. In some examples, the feedback is the quality assessment. In certain examples, the feedback is a quality score. In various examples, the feedback includes true or false classification, a translational deviation matrix, a rotational deviation matrix, and/or a translational-rotational deviation matrix.

In various embodiments, the feedback receiving module 26 is configured to receive a feedback (e.g., feedback generated by the feedback generating module 24), such as by the positioning neural network (e.g., neural network trained for positioning). In certain examples, the feedback receiving module 26 is configured to receive the feedback from the quality assessment neural network (e.g., neural network trained for quality assessment). In various examples, the feedback receiving module 26 is configured to transfer or direct a feedback (e.g., feedback generated by the feedback generating module 24) from a quality assessment neural network to a positioning neural network.

In various embodiments, the parameter changing module 28 is configured to change one or more parameters of a positioning neural network (e.g., neural network trained for positioning) based at least in part on a feedback (e.g., feedback generated by the feedback generating module 24). In some examples, the parameter changing module 28 is part of the positioning neural network and/or is configured to repeatedly change one or more parameters of the positioning neural network. For example, if the quality assessment generated by the quality assessment neural network is unsatisfactory (e.g., when compared to a quality threshold), following the changing of one or more parameters of the positioning neural network, the instruction determining module 14 is further configured to determine an updated positioning instruction by the positioning neural network, the image acquiring module 16 is further configured to acquire a substitute image, the image receiving module 18 is configured to receive the substitute image, the feature identifying module 20 is configured to identify one or more updated features from the substitute image, the quality assessment module 22 is configured to determine an updated quality assessment corresponding to the substitute image, and if the updated quality assessment is unsatisfactory (e.g., when compared to the quality threshold), the parameter changing module 28 is further configured to change one or more parameters of the positioning neural network.

In various embodiments, the unwanted object module 30 is configured to prompt a removal instruction (e.g., to a user) for removing an unwanted object. For example, the unwanted object module 30 is configured to, if the one or more features identified by the feature identifying module 20 includes an unwanted object, prompt the removal instruction for removing an unwanted object. In some examples, the unwanted object module 30 is configured to, such as by controlling the image acquiring module 16, acquire a substitute image. In certain examples, the unwanted object module 30 is configured to acquire the substitute image according to a positioning instruction and a scanning protocol, such as after the unwanted object is removed. In various examples, the unwanted object module 30 is configured to determine a substitute positioning instruction and acquiring a substitute image according to the substitute positioning instruction with the unwanted object circumvented. In some examples, a substitute image is referred to as a replacement image.

In various embodiments, the training module 32 is configured to train the quality assessment neural network (e.g., a neural network trained or to-be-trained for quality assessment). In some examples, the training module 32 is configured to receive a training medical image by the quality assessment neural network. For example, the training module 32 is configured to input the training medical image into the quality assessment neural network. In certain examples, the training module 32 is configured to receive a target output (e.g., a ground truth) associated with the training medical image. In various examples, the training module 32 is configured to, such as by using the quality assessment neural network, generate a training assessment (e.g., a training score) associated with the training medical image. In some examples, the training module 32 is configured to generate a training feedback based at least in part on the training assessment and/or the target output. In certain examples, the training module 32 is configured to change one or more parameters of the quality assessment neural network based at least in part on the training feedback. In some examples, the training feedback includes a true or false classification, a translational deviation matrix, a rotational deviation matrix, and/or a translational-rotational deviation matrix (e.g., one including translational and/or rotational elements). In certain examples, the training module 32 is configured to determine a loss based at least in part on the training assessment and/or the target output. In various examples, the training module 32 is configured to change one or more parameters based at least in part on the loss using a gradient-descension-based machine learning framework.

In various embodiments, the image selecting module 34 is configured to select an image to be the medical image, such as the medical image to be outputted to a display. In certain examples, the image selecting module 34 is configured to, if a quality assessment (e.g., one generated by the quality assessment module 22) satisfies a predetermined quality threshold, select the image corresponding to the quality assessment to be the medical image. In certain examples, the image selecting module 34 is configured to, if the quality assessment does not satisfy a predetermined quality threshold, determine the image corresponding to the quality assessment to be an image not qualified to be selected as the medical image.

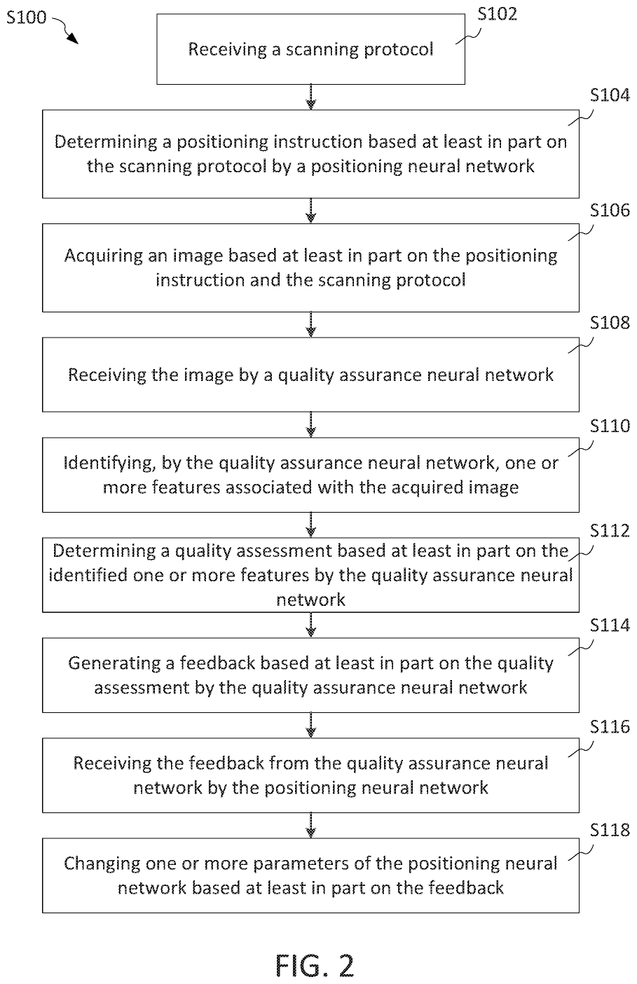

FIG. 2 is a simplified diagram showing a method for using a medical imaging apparatus for acquiring a medical image of a patient, according to some embodiments of the present invention. This diagram is merely an example, which should not unduly limit the scope of the claims. One of ordinary skill in the art would recognize many variations, alternatives, and modifications. In some examples, a method S100 includes a process S102 of receiving a scanning protocol, a process S104 of determining a positioning instruction by a positioning neural network, a process S106 of acquiring an image, a process S108 of receiving the image by a quality assessment neural network, a process S110 of identifying one or more features by the quality assessment neural network, a process S112 of determining a quality assessment by the quality assessment neural network, a process S114 of generating a feedback by the quality assessment neural network, a process S116 of receiving the feedback by the positioning neural network, and a process S118 of changing one or more parameters of the positioning neural network. Although the above has been shown using a selected group of processes for the method, there can be many alternatives, modifications, and variations. For example, some of the processes may be expanded and/or combined. Other processes may be inserted to those noted above. Depending upon the embodiment, the sequence of processes may be interchanged with others replaced.

In various embodiments, the process S102 of receiving a scanning protocol includes receiving a scan type, a patient body type, a target body part, a sampling mask, a magnification, a working distance, a resolution, and/or a scanning rate. In some examples, receiving the scanning protocol includes selecting the scanning protocol from a menu, such as via a user interface.

In various embodiments, the process S104 of determining a positioning instruction by a positioning neural network (e.g., a neural network trained for positioning an object) includes determining the positioning instruction based at least in part on the scanning protocol. In certain examples, determining the positioning instruction includes determining the positioning instruction based at least in part on a relative position between a patient position (e.g., a position of a target object) and a reference position. For example, determining the positioning instruction includes determining a guidance for adjusting the position of an imaging system (e.g., a scanning probe) and/or determining a guidance for adjusting the position of the patient or a part of the patient. In some examples, determining the positioning instruction includes acquiring the patient position, such as based at least in part of an acquired patient image. In certain examples, determining the positioning instruction includes selecting the reference position based at least in part on the scanning protocol and/or patient information. In some examples, determining the positioning instruction includes determining a target region based at least in part on the scanning protocol. For example, determining the positioning instruction includes determining a body part and/or a body organ. In certain examples, the determining the positioning instruction includes determining a scanning technique (e.g., based at least in part on the scanning protocol) and determining a scanning path based at least in part on the scanning technique.

In various embodiments, the process S106 of acquiring an image includes acquiring the image based at least in part on the positioning instruction and/or the scanning protocol. In certain examples, acquiring the image includes sending the positioning instruction to a medical imaging apparatus (e.g., a scanning machine), such as to a positioning system (e.g., a robotic scanning platform and/or a robotic arm) of the medical imaging apparatus, for positioning a target (e.g., a patient). In some examples, acquiring an image includes sending an imaging instruction to an imaging system (e.g., a scanning probe) to acquire an image according to the scanning protocol. In various examples, acquiring the image includes acquiring the image by selecting the image from a pre-generated image database including one or more images previously acquired.

In various embodiments, the process S108 of receiving the image by a quality assessment neural network includes inputting the image into the quality assessment neural network for quality assessment.

In various embodiments, the process S110 of identifying one or more features by the quality assessment neural network includes identifying one or more features associated with an image, such as an image received by the quality assessment neural network. In some examples, identifying one or more features includes extracting one or more features associated with the image by the quality assessment neural network. In certain examples, identifying one or more features includes identifying a landmark, a visual feature, a geometric shape, and/or an unwanted object.

In various embodiments, the process S112 of determining a quality assessment by the quality assessment neural network includes determining the quality assessment based at least in part on one or more features (e.g., one or more features identified by a feature extracting neural network and/or the quality assessment neural network). In some examples, determining the quality assessment includes comparing the identified one or more features against a target feature list and identifying one or more missing features. In certain examples, determining the quality assessment includes determining the quality assessment based at least in part of the identified one or more missing features.

In various embodiments, the process S114 of generating a feedback by the quality assessment neural network includes generating the feedback based at least in part on a quality assessment. In some examples, generating the feedback includes using the quality assessment as the feedback. In certain examples, generating the feedback includes generating a quality score. In various examples, generating the feedback includes generating a true or false classification, a translational deviation matrix, a rotational deviation matrix, and/or a translational-rotational deviation matrix.

In various embodiments, the process S116 of receiving the feedback by the positioning neural network includes receiving the feedback from the quality assessment neural network (e.g., neural network trained for quality assessment). In various examples, receiving the feedback includes transferring or directing the feedback from the quality assessment neural network to the positioning neural network.

In various embodiments, the process S118 of changing one or more parameters of the positioning neural network includes changing one or more parameters of the positioning neural network based at least in part on a feedback and/or a quality assessment. In some examples, one or more of processes S102, S104, S106, S108, S110, S112, S114, S116, and S118 is repeated. For example, if the quality assessment generated by the quality assessment neural network is unsatisfactory (e.g., when compared to a quality threshold), following the changing of one or more parameters of the positioning neural network, the method S100 includes determining an updated positioning instruction by the positioning neural network, acquiring a substitute image, receiving the substitute image, identifying one or more updated features from the substitute image, determining an updated quality assessment corresponding to the substitute image, and if the updated quality assessment is unsatisfactory (e.g., when compared to the quality threshold), changing one or more parameters of the positioning neural network.

In certain embodiments, the method S100 further includes prompting a removal instruction (e.g., to a user) for removing an unwanted object. For example, prompting the removal instruction is performed if one or more features identified (e.g., by the quality assessment module) includes an unwanted object. In some examples, the method S100 further includes acquiring a substitute image, such as after the removal of the unwanted object. In certain examples, the method S100 further includes determining a substitute positioning instruction and acquiring the substitute image according to the substitute positioning instruction with the unwanted object circumvented.

In certain embodiments, the method S100 includes, such as before the receiving a scanning protocol is performed, training the quality assessment neural network. In some examples, training the quality assessment neural network includes receiving a training medical image by the quality assessment neural network, receiving a target output associated with the training medical image, generating a training assessment associated with the training medical image quality assessment neural network, generating the training feedback based at least in part on the training assessment and the target output, and changing one or more parameters of the quality assessment neural network based at least in part on the training feedback. In some examples, generating the training feedback includes generating a true or false classification, a translational-rotational deviation matrix, translational deviation matrix, and/or a rotational deviation matrix. In various examples, generating a training feedback includes determining a loss based at least in part on the training assessment and the target output. In certain examples, changing one or more second parameters of the quality assessment neural network includes changing the one or more parameters based at least in part on the loss using a gradient-descension-based machine learning framework.

In certain embodiments, the method S100 includes selecting an image to be the medical image, such as the medical image to be outputted to a display. In certain examples, selecting the image includes, if a quality assessment satisfies a predetermined quality threshold, selecting the image corresponding to the quality assessment to be the medical image.

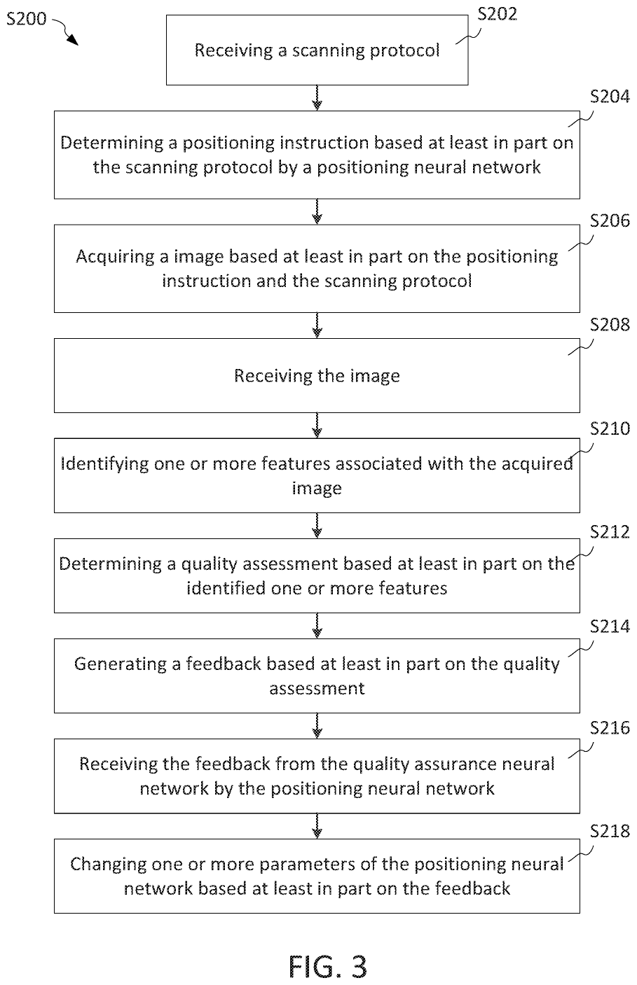

FIG. 3 is a simplified diagram showing a method for using a medical imaging apparatus for acquiring a medical image of a patient, according to some embodiments of the present invention. This diagram is merely an example, which should not unduly limit the scope of the claims. One of ordinary skill in the art would recognize many variations, alternatives, and modifications. In some examples, a method S200 includes a process S202 of receiving a scanning protocol, a process S204 of determining a positioning instruction by a positioning neural network, a process S206 of acquiring an image, a process S208 of receiving the image, a process S210 of identifying one or more features, a process S212 of determining a quality assessment, a process S214 of generating a feedback, a process S216 of receiving the feedback by the positioning neural network, and a process S218 of changing one or more parameters of the positioning neural network. Although the above has been shown using a selected group of processes for the method, there can be many alternatives, modifications, and variations. For example, some of the processes may be expanded and/or combined. Other processes may be inserted to those noted above. Depending upon the embodiment, the sequence of processes may be interchanged with others replaced.

In various embodiments, the process S202 of receiving a scanning protocol includes receiving a scan type, a patient body type, a target body part, a sampling mask, a magnification, a working distance, a resolution, and/or a scanning rate. In some examples, receiving the scanning protocol includes selecting the scanning protocol from a menu, such as via a user interface.

In various embodiments, the process S204 of determining a positioning instruction by a positioning neural network (e.g., a neural network trained for positioning an object) includes determining the positioning instruction based at least in part on the scanning protocol. In certain examples, determining the positioning instruction includes determining the positioning instruction based at least in part on a relative position between a patient position (e.g., a position of a target object) and a reference position. For example, determining the positioning instruction includes determining a guidance for adjusting the position of an imaging system (e.g., a scanning probe) and/or determining a guidance for adjusting the position of the patient or a part of the patient. In some examples, determining the positioning instruction includes acquiring the patient position, such as based at least in part of an acquired patient image. In certain examples, determining the positioning instruction includes selecting the reference position based at least in part on the scanning protocol and/or patient information. In some examples, determining the positioning instruction includes determining a target region based at least in part on the scanning protocol. For example, determining the positioning instruction includes determining a body part and/or a body organ. In certain examples, the determining the positioning instruction includes determining a scanning technique (e.g., based at least in part on the scanning protocol) and determining a scanning path based at least in part on the scanning technique.

In various embodiments, the process S206 of acquiring an image includes acquiring the image based at least in part on the positioning instruction and/or the scanning protocol. In certain examples, acquiring the image includes sending the positioning instruction to a medical imaging apparatus (e.g., a scanning machine), such as to a positioning system (e.g., a robotic scanning platform and/or a robotic arm) of the medical imaging apparatus, for positioning a target (e.g., a patient). In some examples, acquiring an image includes sending an imaging instruction to an imaging system (e.g., a scanning probe) to acquire an image according to the scanning protocol. In various examples, acquiring the image includes acquiring the image by selecting the image from a pre-generated image database including one or more images previously acquired.

In various embodiments, the process S208 of receiving the image includes receiving the image by a user, such as a specialist, a doctor, and/or a medical staff.

In various embodiments, the process S210 of identifying one or more features includes identifying one or more features at least partly by the user. In some examples, identifying one or more features at least partly by the user includes identifying one or more features associated with an image, such as an image received by the user. In some examples, identifying one or more features includes annotating one or more features associated with the image at least partly by the user. In certain examples, identifying one or more features includes identifying a landmark, a visual feature, a geometric shape, and/or an unwanted object.

In various embodiments, the process S212 of determining a quality assessment includes determining the quality assessment at least partly by the user. In some examples, determining the quality assessment at least partly by the user includes determining the quality assessment based at least in part on one or more features (e.g., one or more features identified at least partly by the user). In some examples, determining the quality assessment includes comparing, at least partly by the user, the identified one or more features against a target feature list and identifying one or more missing features. In certain examples, determining the quality assessment includes determining, at least partly by the user, the quality assessment based at least in part of the identified one or more missing features.

In various embodiments, the process S214 of generating a feedback includes generating the feedback at least partly by the user. In some examples, generating the feedback at least partly by the user generating the feedback based at least in part on a quality assessment at least partly by the user. In some examples, generating the feedback includes using the quality assessment as the feedback. In certain examples, generating the feedback includes generating a quality score at least partly by the user. In various examples, generating the feedback includes generating, at least partly by the user, a true or false classification, a translational deviation matrix, a rotational deviation matrix, and/or a translational-rotational deviation matrix.

In various embodiments, the process S216 of receiving the feedback by the positioning neural network includes receiving the feedback from the quality assessment neural network (e.g., neural network trained for quality assessment). In various examples, receiving the feedback includes transferring or directing the feedback from the quality assessment neural network to the positioning neural network.

In various embodiments, the process S218 of changing one or more parameters of the positioning neural network includes changing one or more parameters of the positioning neural network based at least in part on a feedback and/or a quality assessment. In some examples, one or more of processes S202, S204, S206, S208, S210, S212, S214, S216, and S218 is repeated. For example, if the quality assessment generated by the quality assessment neural network is unsatisfactory (e.g., when compared to a quality threshold), following the changing of one or more parameters of the positioning neural network, the method S200 includes determining an updated positioning instruction by the positioning neural network, acquiring a substitute image, receiving the substitute image, identifying one or more updated features from the substitute image, determining an updated quality assessment corresponding to the substitute image, and if the updated quality assessment is unsatisfactory (e.g., when compared to the quality threshold), changing one or more parameters of the positioning neural network.

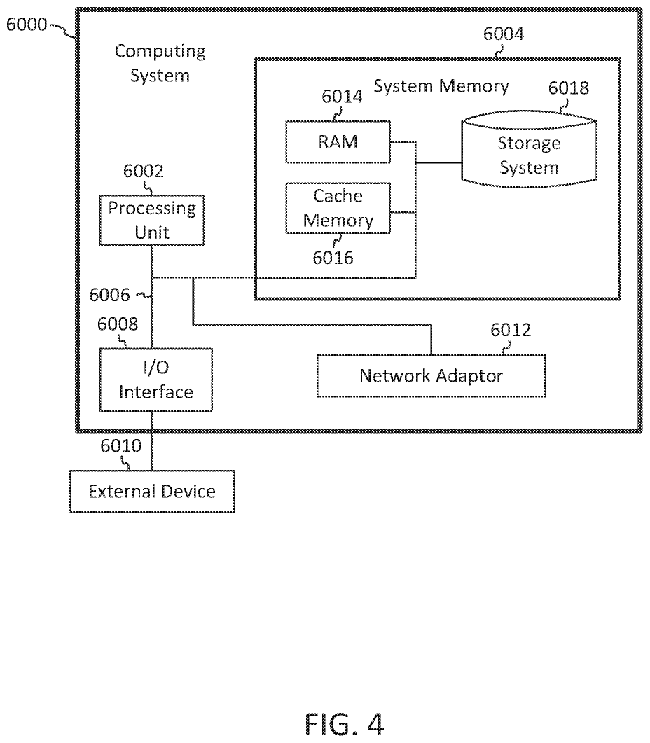

FIG. 4 is a simplified diagram showing a computing system, according to some embodiments. This diagram is merely an example, which should not unduly limit the scope of the claims. One of ordinary skill in the art would recognize many variations, alternatives, and modifications. In certain examples, the computing system 6000 is a general-purpose computing device. In some examples, the computing system 6000 includes one or more processing units 6002 (e.g., one or more processors), one or more system memories 6004, one or more buses 6006, one or more input/output (I/O) interfaces 6008, and/or one or more network adapters 6012. In certain examples, the one or more buses 6006 connect various system components including, for example, the one or more system memories 6004, the one or more processing units 6002, the one or more input/output (I/O) interfaces 6008, and/or the one or more network adapters 6012. Although the above has been shown using a selected group of components for the computing system, there can be many alternatives, modifications, and variations. For example, some of the components may be expanded and/or combined. Other components may be inserted to those noted above. Depending upon the embodiment, the arrangement of components may be interchanged with others replaced.

In certain examples, the computing system 6000 is a computer (e.g., a server computer, a client computer), a smartphone, a tablet, or a wearable device. In some examples, some or all processes (e.g., steps) of the method S100 and/or the method S200 are performed by the computing system 6000. In certain examples, some or all processes (e.g., steps) of the method S100 and/or the method S200 are performed by the one or more processing units 6002 directed by one or more codes. For example, the one or more codes are stored in the one or more system memories 6004 (e.g., one or more non-transitory computer-readable media), and are readable by the computing system 6000 (e.g., readable by the one or more processing units 6002). In various examples, the one or more system memories 6004 include one or more computer-readable media in the form of volatile memory, such as a random-access memory (RAM) 6014, a cache memory 6016, and/or a storage system 6018 (e.g., a floppy disk, a CD-ROM, and/or a DVD-ROM).

In some examples, the one or more input/output (I/O) interfaces 6008 of the computing system 6000 is configured to be in communication with one or more external devices 6010 (e.g., a keyboard, a pointing device, and/or a display). In certain examples, the one or more network adapters 6012 of the computing system 6000 is configured to communicate with one or more networks (e.g., a local area network (LAN), a wide area network (WAN), and/or a public network (e.g., the Internet)). In various examples, additional hardware and/or software modules are utilized in connection with the computing system 6000, such as one or more micro-codes and/or one or more device drivers.

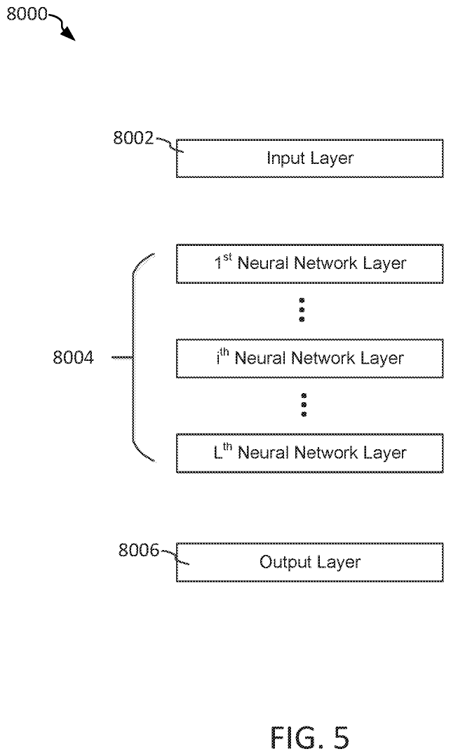

FIG. 5 is a simplified diagram showing a neural network, according to certain embodiments. This diagram is merely an example, which should not unduly limit the scope of the claims. One of ordinary skill in the art would recognize many variations, alternatives, and modifications. The neural network 8000 is an artificial neural network. In some examples, the neural network 8000 includes an input layer 8002, one or more hidden layers 8004, and an output layer 8006. For example, the one or more hidden layers 8004 includes L number of neural network layers, which include a 1^{st} neural network layer, . . . , an i^{th} neural network layer, . . . and an L^{th} neural network layer, where L is a positive integer and i is an integer that is larger than or equal to 1 and smaller than or equal to L. Although the above has been shown using a selected group of components for the neural network, there can be many alternatives, modifications, and variations. For example, some of the components may be expanded and/or combined. Other components may be inserted to those noted above. Depending upon the embodiment, the arrangement of components may be interchanged with others replaced.

In some examples, some or all processes (e.g., steps) of the method S100 and/or the method S200 are performed by the neural network 8000 (e.g., using the computing system 6000). In certain examples, some or all processes (e.g., steps) of the method S100 and/or the method S200 are performed by the one or more processing units 6002 directed by one or more codes that implement the neural network 8000. For example, the one or more codes for the neural network 8000 are stored in the one or more system memories 6004 (e.g., one or more non-transitory computer-readable media), and are readable by the computing system 6000 such as by the one or more processing units 6002.

In certain examples, the neural network 8000 is a deep neural network (e.g., a convolutional neural network). In some examples, each neural network layer of the one or more hidden layers 8004 includes multiple sublayers. As an example, the i^{th} neural network layer includes a convolutional layer, an activation layer, and a pooling layer. For example, the convolutional layer is configured to perform feature extraction on an input (e.g., received by the input layer or from a previous neural network layer), the activation layer is configured to apply a nonlinear activation function (e.g., a ReLU function) to the output of the convolutional layer, and the pooling layer is configured to compress (e.g., to down-sample, such as by performing max pooling or average pooling) the output of the activation layer. As an example, the output layer 8006 includes one or more fully connected layers.

As discussed above and further emphasized here, FIG. 5 is merely an example, which should not unduly limit the scope of the claims. One of ordinary skill in the art would recognize many variations, alternatives, and modifications. For example, the neural network 8000 is replaced by an algorithm that is not an artificial neural network. As an example, the neural network 8000 is replaced by a model for machine learning that is not an artificial neural network.

In various embodiments, a computer-implemented method for using a medical imaging apparatus for acquiring a medical image of a patient includes: receiving a scanning protocol; determining a first positioning instruction based at least in part on the scanning protocol by a first neural network, the first neural network being previously-trained for positioning; acquiring a first image based at least in part on the first positioning instruction and the scanning protocol; receiving the first image (e.g., by a second neural network previously-trained for quality assessment); identifying (e.g., by the second neural network) one or more first features associated with the acquired first image; determining a first quality assessment based at least in part on the identified one or more first features (e.g., by the second neural network), the first quality assessment being associated with the first image; generating a first feedback based at least in part on the first quality assessment (e.g., by the second neural network); receiving the first feedback (e.g., from the second neural network) by the first neural network; and changing one or more first parameters of the previously-trained first neural network based at least in part on the first feedback. In certain examples, the computer-implemented method is performed by one or more processors. In some examples, the computer-implemented method is implemented according to at least the method S100 of FIG. 2 and/or the method S200 of FIG. 3. In certain examples, the method is implemented by at least the system 10 of FIG. 1.

In some embodiments, the computer-implemented method further includes: if the first quality assessment satisfies a predetermined quality threshold, selecting the first image as the medical image; and if the first quality assessment fails to satisfy the predetermined quality threshold: determining a second positioning instruction using the first neural network with the changed one or more first parameters; acquiring a second image according to the second positioning instruction and the scanning protocol; receiving the second image (e.g., by the second neural network); identifying one or more second features associated with the acquired second image (e.g., by the second neural network); determining a second quality assessment associated with the second image based at least in part on the identified one or more second features (e.g., by the second neural network); and if the second quality assessment satisfies the predetermined quality threshold, selecting the second image as the medical image.

In some embodiments, the computer-implemented method further includes: before the receiving a scanning protocol is performed, training a second neural network; wherein the training a second neural network for quality assessment includes: receiving a training medical image by the second neural network; receiving a target output associated with the training medical image; generating a training assessment associated with the training medical image by the second neural network; generating the training feedback based at least in part on the training assessment and the target output; and changing one or more second parameters of the second neural network based at least in part on the training feedback.

In some embodiments, the training feedback includes a true or false classification, a translational-rotational deviation matrix, translational deviation matrix, and/or a rotational deviation matrix.

In some embodiments, the generating a training feedback includes determining a loss based at least in part on the training assessment and the target output; and the changing one or more second parameters of the second neural network includes changing the one or more second parameters based at least in part on the loss using a gradient-descension-based machine learning framework.

In some embodiments, receiving a scanning protocol includes receiving a scan type, a patient body type, a target body part, a sampling mask, a magnification, a working distance, a resolution, and/or a scanning rate.

In some embodiments, determining a first positioning instruction based at least in part on the scanning protocol by a first neural network includes: determining a target region based at least in part on the scanning protocol; determining a scanning technique for scanning the target region; and determining a first scanning path based at least in part on the scanning technique.

In some embodiments, acquiring a first image based at least in part on the first positioning instruction and the scanning protocol includes: sending the first positioning instruction to a positioning system of the medical imaging apparatus for positioning the patient to a first relative position relative to an imaging system of the medical imaging apparatus; and sending an imaging instruction to the imaging system to acquire the first image according to the scanning protocol.

In some embodiments, identifying one or more first features associated with the acquired first image (e.g., by the second neural network) includes identifying a landmark, a visual feature, a geometric shape, and/or an unwanted object.

In some embodiments, if the one or more first features includes an unwanted object, the method further includes: prompting a removal instruction for removing the unwanted object and acquiring a first substitute image according to the first positioning instruction and the scanning protocol; and/or determining a second positioning instruction and acquiring a second image according to the second positioning instruction with the unwanted object circumvented.

In some embodiments, determining a first quality assessment associated with the first image based at least in part on the identified one or more first features (e.g., by the second neural network) includes comparing the identified one or more first features against a target feature list and identifying one or more missing features.

In various embodiments, a system for using a medical imaging apparatus for acquiring a medical image of a patient includes: a protocol receiving module configured to receive a scanning protocol; an instruction determining module configured to determine a first positioning instruction based at least in part on the scanning protocol by a first neural network, the first neural network being previously-trained for positioning; an image acquiring module configured to acquire a first image based at least in part on the first positioning instruction and the scanning protocol; an image receiving module configured to receive the first image (e.g., by a second neural network previously-trained for quality assessment); a feature identifying module configured to identify (e.g., by the second neural network) one or more first features associated with the acquired first image; a quality assessment module configured to determine a first quality assessment based at least in part on the identified one or more first features (e.g., by the second neural network) the first quality assessment being associated with the first image; a feedback generating module configured to generate a first feedback based at least in part on the first quality assessment (e.g., by the second neural network); a feedback receiving module configured to receive the first feedback (e.g., from the second neural network) by the first neural network; and a parameter changing module configured to change one or more first parameters of the previously-trained first neural network based at least in part on the first feedback. In some examples, the system is implemented according to at least the system 10 of FIG. 1 and/or configured to perform at least the method S100 of FIG. 2 and/or the method S200 of FIG. 3.

In some embodiments, the system further includes: an image selecting module configured to, if the first quality assessment satisfies a predetermined quality threshold, select the first image as the medical image. In certain examples, if the first quality assessment fails to satisfy the predetermined quality threshold: the instruction determining module is further configured to determine a second positioning instruction using the first neural network with the changed one or more first parameters; the image acquiring module is further configured to acquire a second image according to the second positioning instruction and the scanning protocol; the image receiving module is further configured to receive the second image (e.g., by the second neural network); the feature identifying module is further configured to identify one or more second features associated with the acquired second image (e.g., by the second neural network); the quality assessment module is further configured to determine a second quality assessment associated with the second image based at least in part on the identified one or more second features (e.g., by the second neural network); and the image selecting module is further configured to, if the second quality assessment satisfies the predetermined quality threshold, select the second image as the medical image.

In some embodiments, the system further includes: a training module configured to train a second neural network, the training module configured to: receive a training medical image by the second neural network; receive a target output associated with the training medical image; generate a training assessment associated with the training medical image by the second neural network; generate the training feedback based at least in part on the training assessment and the target output; and change one or more second parameters of the second neural network based at least in part on the training feedback.

In some embodiments, the training feedback includes a true or false classification, a translational deviation matrix, a rotational deviation matrix, and/or a translational-rotational deviation matrix.

In some embodiments, the training module is further configured to: determine a loss based at least in part on the training assessment and the target output; and change the one or more second parameters based at least in part on the loss using a gradient-descension-based machine learning framework.

In some embodiments, the scanning protocol includes a scan type, a patient body type, a target body part, a sampling mask, a magnification, a working distance, a resolution, and/or a scanning rate.

In some embodiments, the instruction determining module is further configured to determine a target region based at least in part on the scanning protocol; determine a scanning technique for scanning the target region; and determine a first scanning path based at least in part on the scanning technique.

In some embodiments, the image acquiring module is further configured to: send the first positioning instruction to a positioning system of the medical imaging apparatus for positioning the patient to a first relative position relative to an imaging system of the medical imaging apparatus; and send an imaging instruction to the imaging system to acquire the first image according to the scanning protocol.

In some embodiments, the feature identifying module is further configured to identify a landmark, a visual feature, a geometric shape, and/or an unwanted object.

In some embodiments, the system further includes an unwanted object module configured to, if the one or more first features includes an unwanted object, prompt a removal instruction for removing the unwanted object and acquire a first substitute image according to the first positioning instruction and the scanning protocol; and/or determine a second positioning instruction and acquiring a second image according to the second positioning instruction with the unwanted object circumvented.

In some embodiments, the quality assessment module is further configured to compare the identified one or more first features against a target feature list and identify one or more missing features.

In various embodiments, a non-transitory computer-readable medium with instructions stored thereon, that when executed by a processor, perform the processes including: receiving a scanning protocol; determining a first positioning instruction based at least in part on the scanning protocol by a first neural network, the first neural network being previously-trained for positioning; acquiring a first image based at least in part on the first positioning instruction and the scanning protocol; receiving the first image (e.g., by a second neural network previously-trained for quality assessment); identifying (e.g., by the second neural network) one or more first features associated with the acquired first image; determining a first quality assessment based at least in part on the identified one or more first features (e.g., by the second neural network), the first quality assessment being associated with the first image; generating a first feedback based at least in part on the first quality assessment (e.g., by the second neural network); receiving the first feedback from the second neural network by the first neural network; and changing one or more first parameters of the previously-trained first neural network based at least in part on the first feedback. In some examples, the non-transitory computer-readable medium with instructions stored thereon is implemented according to at least the method S100 of FIG. 2, and/or by the system 10 (e.g., a terminal) of FIG. 1.

In some embodiments, the non-transitory computer-readable medium, that when executed by a processor, further perform the processes including: if the first quality assessment satisfies a predetermined quality threshold, selecting the first image as the medical image; and if the first quality assessment fails to satisfy the predetermined quality threshold: determining a second positioning instruction using the first neural network with the changed one or more first parameters; acquiring a second image according to the second positioning instruction and the scanning protocol; receiving the second image (e.g., by the second neural network); identifying one or more second features associated with the acquired second image (e.g., by the second neural network); determining a second quality assessment associated with the second image based at least in part on the identified one or more second features (e.g., by the second neural network); and if the second quality assessment satisfies the predetermined quality threshold, selecting the second image as the medical image.

In some embodiments, the non-transitory computer-readable medium, that when executed by a processor, further perform the processes including: before the receiving a scanning protocol is performed, training a second neural network; wherein the training a second neural network for quality assessment includes: receiving a training medical image by the second neural network; receiving a target output associated with the training medical image; generating a training assessment associated with the training medical image by the second neural network; generating the training feedback based at least in part on the training assessment and the target output; and changing one or more second parameters of the second neural network based at least in part on the training feedback.

In some embodiments, the training feedback includes a true or false classification, a translational-rotational deviation matrix, translational deviation matrix, and/or a rotational deviation matrix.

In some embodiments, the non-transitory computer-readable medium, that when executed by a processor, perform the processes including: determining a loss based at least in part on the training assessment and the target output; and the changing one or more second parameters of the second neural network includes changing the one or more second parameters based at least in part on the loss using a gradient-descension-based machine learning framework.

In some embodiments, the non-transitory computer-readable medium, that when executed by a processor, perform the processes including: receiving a scan type, a patient body type, a target body part, a sampling mask, a magnification, a working distance, a resolution, and/or a scanning rate.

In some embodiments, the non-transitory computer-readable medium, that when executed by a processor, perform the processes including: determining a target region based at least in part on the scanning protocol; determining a scanning technique for scanning the target region; and determining a first scanning path based at least in part on the scanning technique.

In some embodiments, the non-transitory computer-readable medium, that when executed by a processor, perform the processes including: sending the first positioning instruction to a positioning system of the medical imaging apparatus for positioning the patient to a first relative position relative to an imaging system of the medical imaging apparatus; and sending an imaging instruction to the imaging system to acquire the first image according to the scanning protocol.

In some embodiments, the non-transitory computer-readable medium, that when executed by a processor, perform the processes including: identifying a landmark, a visual feature, a geometric shape, and/or an unwanted object.

In some embodiments, if the one or more first features includes an unwanted object, the non-transitory computer-readable medium, that when executed by a processor, further perform the processes including: prompting a removal instruction for removing the unwanted object and acquiring a first substitute image according to the first positioning instruction and the scanning protocol; and/or determining a second positioning instruction and acquiring a second image according to the second positioning instruction with the unwanted object circumvented.

In some embodiments, the non-transitory computer-readable medium, that when executed by a processor, perform the processes including: comparing the identified one or more first features against a target feature list and identifying one or more missing features.

In various embodiments, a method for using a medical imaging apparatus for acquiring a medical image of a patient includes: receiving a scanning protocol; determining a first positioning instruction based at least in part on the scanning protocol by a first neural network, the first neural network being previously-trained for positioning; acquiring a first image based at least in part on the first positioning instruction and the scanning protocol; receiving the first image; identifying one or more first features associated with the acquired first image; determining a first quality assessment based at least in part on the identified one or more first features, the first quality assessment being associated with the first image; generating a first feedback based at least in part on the first quality assessment; receiving the first feedback by the first neural network; and changing, by the first neural network, one or more first parameters of the previously-trained first neural network based at least in part on the first feedback.

In some embodiments, the method further includes: identifying, by a second neural network, one or more second features associated with the acquired first image, the second neural network being previously-trained for quality assessment; determining a second quality assessment based at least in part on the identified one or more second features by the second neural network, the second quality assessment being associated with the first image; generating a second feedback based at least in part on the second quality assessment by the second neural network; receiving the first feedback by the second neural network; and changing, by the second neural network, one or more second parameters of the previously-trained second neural network based at least in part on the received first feedback and the determined second feedback.

For example, some or all components of various embodiments of the present invention each are, individually and/or in combination with at least another component, implemented using one or more software components, one or more hardware components, and/or one or more combinations of software and hardware components. In another example, some or all components of various embodiments of the present invention each are, individually and/or in combination with at least another component, implemented in one or more circuits, such as one or more analog circuits and/or one or more digital circuits. In yet another example, while the embodiments described above refer to particular features, the scope of the present invention also includes embodiments having different combinations of features and embodiments that do not include all of the described features. In yet another example, various embodiments and/or examples of the present invention can be combined.

Additionally, the methods and systems described herein may be implemented on many different types of processing devices by program code including program instructions that are executable by the device processing subsystem. The software program instructions may include source code, object code, machine code, or any other stored data that is operable to cause a processing system to perform the methods and operations described herein. Other implementations may also be used, however, such as firmware or even appropriately designed hardware configured to perform the methods and systems described herein.

The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, EEPROM, Flash memory, flat files, databases, programming data structures, programming variables, IF-THEN (or similar type) statement constructs, application programming interface, etc.). It is noted that data structures describe formats for use in organizing and storing data in databases, programs, memory, or other computer-readable media for use by a computer program.

The systems and methods may be provided on many different types of computer-readable media including computer storage mechanisms (e.g., CD-ROM, diskette, RAM, flash memory, computer's hard drive, DVD, etc.) that contain instructions (e.g., software) for use in execution by a processor to perform the methods' operations and implement the systems described herein. The computer components, software modules, functions, data stores and data structures described herein may be connected directly or indirectly to each other in order to allow the flow of data needed for their operations. It is also noted that a module or processor includes a unit of code that performs a software operation and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

The computing system can include client devices and servers. A client device and server are generally remote from each other and typically interact through a communication network. The relationship of client device and server arises by virtue of computer programs running on the respective computers and having a client device-server relationship to each other.

This specification contains many specifics for particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations, one or more features from a combination can in some cases be removed from the combination, and a combination may, for example, be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Although specific embodiments of the present invention have been described, it will be understood by those of skill in the art that there are other embodiments that are equivalent to the described embodiments. Accordingly, it is to be understood that the invention is not to be limited by the specific illustrated embodiments.

What is claimed is:

1. A computer-implemented method for using a medical imaging apparatus for acquiring a medical image of a patient, the method comprising:
receiving a scanning protocol;
determining a first positioning instruction based at least in part on the scanning protocol by a first neural network, the first neural network being previously-trained for positioning;
acquiring a first image based at least in part on the first positioning instruction and the scanning protocol;
receiving the first image;
identifying one or more first features associated with the acquired first image;
determining a first quality assessment based at least in part on the identified one or more first features, the first quality assessment being associated with the first image;
generating a first feedback based at least in part on the first quality assessment;
receiving the first feedback by the first neural network; and
changing one or more first parameters of the previously-trained first neural network based at least in part on the first feedback;
wherein the computer-implemented method is performed by one or more processors.

2. The computer-implemented method of claim 1, the method further comprising:
if the first quality assessment satisfies a predetermined quality threshold, selecting the first image as the medical image; and
if the first quality assessment fails to satisfy the predetermined quality threshold:
determining a second positioning instruction using the first neural network with the changed one or more first parameters;
acquiring a second image according to the second positioning instruction and the scanning protocol;
receiving the second image;
identifying one or more second features associated with the acquired second image;
determining a second quality assessment associated with the second image based at least in part on the identified one or more second features; and
if the second quality assessment satisfies the predetermined quality threshold, selecting the second image as the medical image.

3. The computer-implemented method of claim 1, further comprising:
before the receiving a scanning protocol is performed, training a second neural network for quality assessment;
wherein the training a second neural network for quality assessment includes:
receiving a training medical image by the second neural network;
receiving a target output associated with the training medical image;
generating a training assessment associated with the training medical image by the second neural network;
generating the training feedback based at least in part on the training assessment and the target output; and
changing one or more second parameters of the second neural network based at least in part on the training feedback.

4. The computer-implemented method of claim 3, wherein the training feedback includes one selected from a true or false classification, a translational deviation matrix, a rotational deviation matrix, and a translational-rotational deviation matrix.

5. The computer-implemented method of claim 3, wherein:
the generating a training feedback includes determining a loss based at least in part on the training assessment and the target output; and
the changing one or more second parameters of the second neural network includes changing the one or more second parameters based at least in part on the loss using a gradient-descension-based machine learning framework.

6. The computer-implemented method of claim 1, wherein the receiving a scanning protocol includes receiving at least one selected from a scan type, a patient body type, a target body part, a sampling mask, a magnification, a working distance, a resolution, and a scanning rate.

7. The computer-implemented method of claim 1, wherein the determining a first positioning instruction based at least in part on the scanning protocol by a first neural network includes:
determining a target region based at least in part on the scanning protocol;
determining a scanning technique for scanning the target region; and
determining a first scanning path based at least in part on the scanning technique.

8. The computer-implemented method of claim 1, wherein the acquiring a first image based at least in part on the first positioning instruction and the scanning protocol includes:
sending the first positioning instruction to a positioning system of the medical imaging apparatus for positioning the patient to a first relative position relative to an imaging system of the medical imaging apparatus; and
sending an imaging instruction to the imaging system to acquire the first image according to the scanning protocol.

9. The computer-implemented method of claim 1, wherein the identifying one or more first features associated with the acquired first image includes identifying at least one selected from a landmark, a visual feature, a geometric shape, and an unwanted object.

10. The computer-implemented method of claim 9, if the one or more first features includes an unwanted object, the method further comprising one of:
prompting a removal instruction for removing the unwanted object and acquiring a first substitute image according to the first positioning instruction and the scanning protocol; and
determining a second positioning instruction and acquiring a second image according to the second positioning instruction with the unwanted object circumvented.

11. The computer-implemented method of claim 1, wherein the determining a first quality assessment associated with the first image based at least in part on the identified one or more first features includes comparing the identified one or more first features against a target feature list and identifying one or more missing features.

12. A system for computer-implemented method for using a medical imaging apparatus for acquiring a medical image of a patient, the system comprising:
a protocol receiving module configured to receive a scanning protocol;
an instruction determining module configured to determine a first positioning instruction based at least in part on the scanning protocol by a first neural network, the first neural network being previously-trained for positioning;
an image acquiring module configured to acquire a first image based at least in part on the first positioning instruction and the scanning protocol;
an image receiving module configured to receive the first image;
a feature identifying module configured to identify one or more first features associated with the acquired first image;
a quality assessment module configured to determine a first quality assessment based at least in part on the identified one or more first features, the first quality assessment being associated with the first image;
a feedback generating module configured to generate a first feedback based at least in part on the first quality assessment;
a feedback receiving module configured to receive the first feedback by the first neural network; and
a parameter changing module configured to change one or more first parameters of the previously-trained first neural network based at least in part on the first feedback.

13. The system of claim 12, further comprising:
an image selecting module configured to, if the first quality assessment satisfies a predetermined quality threshold, select the first image as the medical image;
wherein if the first quality assessment fails to satisfy the predetermined quality threshold:
the instruction determining module is further configured to determine a second positioning instruction using the first neural network with the changed one or more first parameters;
the image acquiring module is further configured to acquire a second image according to the second positioning instruction and the scanning protocol;
the image receiving module is further configured to receive the second image;
the feature identifying module is further configured to identify one or more second features associated with the acquired second image;
the quality assessment module is further configured to determine a second quality assessment associated with the second image based at least in part on the identified one or more second features; and
the image selecting module is further configured to, if the second quality assessment satisfies the predetermined quality threshold, select the second image as the medical image.

14. The system of claim 12, further comprising:
a training module configured to train a second neural network, the training module configured to:
receive a training medical image by the second neural network;
receive a target output associated with the training medical image;
generate a training assessment associated with the training medical image by the second neural network;
generate the training feedback based at least in part on the training assessment and the target output; and
change one or more second parameters of the second neural network based at least in part on the training feedback.

15. The system of claim 14, wherein the training feedback includes one selected from a true or false classification, a translational deviation matrix, a rotational deviation matrix, and a translational-rotational deviation matrix.

16. The system of claim 14, wherein the training module is further configured to:
determine a loss based at least in part on the training assessment and the target output; and
change the one or more second parameters based at least in part on the loss using a gradient-descension-based machine learning framework.

17. The system of claim 13, wherein the scanning protocol includes at least one selected from a scan type, a patient body type, a target body part, a sampling mask, a magnification, a working distance, a resolution, and a scanning rate.

18. The system of claim 13, wherein the instruction determining module is further configured to:
determine a target region based at least in part on the scanning protocol;
determine a scanning technique for scanning the target region; and
determine a first scanning path based at least in part on the scanning technique.

19. A method for using a medical imaging apparatus for acquiring a medical image of a patient, the method comprising:
receiving a scanning protocol;
determining a first positioning instruction based at least in part on the scanning protocol by a first neural network, the first neural network being previously-trained for positioning;
acquiring a first image based at least in part on the first positioning instruction and the scanning protocol;
receiving the first image;
identifying one or more first features associated with the acquired first image;
determining a first quality assessment based at least in part on the identified one or more first features, the first quality assessment being associated with the first image;
generating a first feedback based at least in part on the first quality assessment;
receiving the first feedback by the first neural network; and
changing, by the first neural network, one or more first parameters of the previously-trained first neural network based at least in part on the first feedback.

20. The method of claim 19, further comprising:
identifying, by a second neural network, one or more second features associated with the acquired first image, the second neural network being previously-trained for quality assessment;
determining a second quality assessment based at least in part on the identified one or more second features by the second neural network, the second quality assessment being associated with the first image;
generating a second feedback based at least in part on the second quality assessment by the second neural network;
receiving the first feedback by the second neural network; and
changing, by the second neural network, one or more second parameters of the previously-trained second neural network based at least in part on the received first feedback and the determined second feedback.
